(19) United States
(12) Patent Application Publication
(10) Pub. No.: US 2024/0351973 A1
(43) Pub. Date: Oct. 24, 2024

(54) METHOD FOR PURIFYING A TEREPHTHALATE DIESTER MONOMER BY ADSORPTION

(71) Applicants: IFP ENERGIES NOUVELLES, RUEIL-MALMAISON (FR); JEPLAN, INC., Kanagawa (JP)

(72) Inventors: David CHICHE, RUEIL-MALMAISON CEDEX (FR); Guillaume BLANCKE, RUEIL-MALMAISON CEDEX (FR); Damien LEINEKUGEL LE COCQ, RUEIL-MALMAISON CEDEX (FR)

(73) Assignees: IFP ENERGIES NOUVELLES, RUEIL-MALMAISON (FR); JEPLAN, INC., Kanagawa (JP)

(21) Appl. No.: 18/571,364

(22) PCT Filed: Jun. 7, 2022
(86) PCT No.: PCT/EP2022/065428

§ 371 Date: Dec. 18, 2023

(30) Foreign Application Priority Data
Jun. 17, 2021 (FR) FR2106439

(51) Int. Cl. C07C 67/56; B01D 9/00; B01D 15/08; B01J 20/20

(52) U.S. Cl. CPC C07C 67/56; B01D 9/005; B01D 15/08; B01J 20/20; B01D 2009/0086

(57) ABSTRACT

The present invention relates to a process for purifying a crude diester monomer feedstock comprising:
a) a step of mixing the crude diester monomer feedstock with an aqueous solvent, at a temperature of between 60° C. and 150° C., in order to obtain a diester monomer aqueous mixture, the amount of aqueous solvent introduced being adjusted so that the crude diester monomer feedstock represents between 20% and 90% of the total weight of the diester monomer aqueous mixture;
b) a step of adsorbing the diester monomer aqueous mixture, at a temperature of between 60° C. and 150° C. and a pressure of between 0.1 and 1.0 MPa, in order to obtain a purified monomer effluent.

### TECHNICAL FIELD

The invention relates to a process for purifying a diester monomer, in particular a terephthalate diester monomer, and in particular bis(2-hydroxyethyl) terephthalate (BHET), by adsorption. More particularly, the invention relates to a process for purifying a crude diester monomer feedstock, in particular comprising a terephthalate diester monomer and notably bis(2-hydroxyethyl) terephthalate (BHET), by adsorption of a mixture of said feedstock with an aqueous solvent by at least one adsorbent, in order to obtain a purified and decolourized diester monomer effluent. The crude diester monomer feedstock may be obtained for example by depolymerization of a polyester feedstock consisting in particular of polyester waste and of post-consumer plastics.

### PRIOR ART

The chemical recycling of polyester, in particular of polyethylene terephthalate (PET), has been the subject of numerous studies aimed at breaking down the polyester, recovered in the form of waste, into monomers which will be able to be used again as feedstock for a polymerization process.

Numerous polyesters result from networks for collecting and sorting materials. In particular, polyester, in particular PET, may originate from the collection of bottles, container trays, films, resins and/or fibres composed of polyester (for instance textile fibres, tyre fibres). The polyester resulting from collecting and sorting channels is referred to as polyester to be recycled.

PET to be recycled can be classified into four main categories:
clear PET, predominantly composed of colourless transparent PET (generally at least 60% by weight) and azure coloured transparent PET, which does not contain pigments and can be used in mechanical recycling processes;
dark or coloured (green, red, etc.) PET, which can generally contain up to 0.1% by weight of dyes or pigments but remains transparent or translucent;
opaque PET, which contains a significant amount of pigments at contents typically varying between 0.25% and 5.0% by weight in order to opacify the polymer. Opaque PET is increasingly being used, for example in the manufacture of food containers, such as milk bottles, in the composition of cosmetic, plant-protection or dye bottles;
multilayer PET, which comprises layers of polymers other than PET or a layer of recycled PET between layers of virgin PET (that is to say, PET which has not undergone recycling), or a film of aluminium, for example. Multilayer PET is used, after thermoforming, to produce packagings, such as container trays.

After collection and sorting, the recycling of these streams generally consists of a first, conditioning step during which bales of raw packaging are washed, purified and sorted, ground and then purified again and sorted to produce a stream of flakes generally containing less than 1% by weight of “macroscopic” impurities (glass, metals, other plastics, wood, paper, cardboard, inorganic elements), preferentially less than 0.2% of “macroscopic” impurities and even more preferentially less than 0.05%.

Clear PET flakes may subsequently undergo an extrusion-filtration step to produce extrudates which can subsequently be reused as a mixture with virgin PET to produce new products (bottles, fibres, films). A step of solid state polymerization (known by the initialism SSP) under vacuum is often necessary for food uses. This type of recycling is known as mechanical recycling.

Dark (or coloured) PET flakes can also be recycled mechanically. However, the colouration of the extrudates formed from the coloured streams limits the uses: dark PET is generally used to produce packaging straps or fibres. The outlets are thus more limited in comparison with those of clear PET.

The presence of opaque PET containing pigments at high contents, in PET to be recycled, presents problems to recyclers as opaque PET adversely affects the mechanical properties of recycled PET. Opaque PET is currently collected with coloured PET and is found in the coloured PET stream. In view of the development of the uses for opaque PET, the contents of opaque PET in the stream of coloured PET to be recycled are currently between 5-20% by weight and are tending to increase further. In a few years' time, it will be possible to achieve contents of opaque PET in the coloured PET stream of greater than 20-30% by weight. However, it has been shown that, above 10-15% of opaque PET in the coloured PET streams, the mechanical properties of the recycled PET are adversely affected (cf. Impact du developpement du PET opaque blanc sur le recyclage des emballages en PET [Impact of the growth of white opaque PET on the recycling of PET packagings], preliminary report of COTREP of 5/12/13) and prevent recycling in the form of fibres, the main outlet of the channel for coloured PET.

Dyes are natural or synthetic substances which are soluble, notably in the polyester material, and are used to colour the material into which they are introduced. The dyes generally used have different natures and often contain heteroatoms of O and N type, and conjugated unsaturations, such as, for example, quinone, methine or azo functions, or molecules such as pyrazolone and quinophthalone.

Pigments are finely divided substances which are insoluble, in particular in the polyester material, and which are used to colour and/or opacify the material into which they are introduced. The main pigments used to colour and/or opacify the polyesters, in particular PET, are metal oxides, such as TiO₂, CoAl₂O₄ or Fe₂O₃, silicates, polysulfides and carbon black. The pigments are particles with a size generally of between 0.1 and 10 μm and predominantly between 0.4 and 0.8 μm. The complete removal of these pigments, which is necessary in order to envisage recycling the opaque PET, by filtration is technically difficult as they have an extremely high clogging capability.

The recycling of coloured and opaque PETs is thus extremely problematic.

Patent application US 2006/0074136 describes a process for depolymerization by glycolysis of coloured PET, in particular resulting from the recovery of green-coloured PET bottles. The PET feedstock treated by this process is contacted with ethylene glycol at a temperature of between 180° C. and 280° C. for several hours. The product of glycolysis which is obtained on conclusion of the depolymerization step is purified, directly or after filtration, over activated carbon at a temperature above 170° C. and then by extraction of the residual dyes, particularly yellow-coloured dyes, with a solvent, which may be an alcohol, such as methanol, or a glycol, such as ethylene glycol, and crystallization of the BHET in the extraction solvent by lowering of the temperature. The BHET is then separated by filtration.

In patent application US 2015/0105532, the post-consumer PET, which comprises a mixture of various PETs, such as clear PET and coloured PETs such as blue PET, green PET and/or amber PET, is depolymerized by glycolysis in the presence of ethylene glycol and an amine catalyst and alcohol, in a reactor at 150-250° C., in batch mode. The resulting diester monomer is purified by direct filtration, then by adsorption on activated carbon and lastly by passage over ion-exchange resin, in particular at a temperature of 80-90° C., before being crystallized and recovered by filtration. Patent application US 2015/0105532 discloses another method for purification of the diester monomer obtained by short-path distillation at 200° C.

U.S. Pat. No. 6,642,350 in turn describes the purification of a solution of crude BHET dissolved in methanol or ethylene glycol, comprising at least successively contacting said solution with an activated carbon, an anion-exchange resin and a cation-exchange resin, at a temperature of between 40° C. and 120° C., in particular of equal to 60° C., 65° C. or 80° C. Specifically, this patent shows that contacting with activated carbon alone under the conditions described above is not sufficient notably to completely decolourize the solution, since a residual colour, in particular yellow, persists, whereas the yellow colouring no longer appears after successively passing over activated carbon and anion- and cation-exchange resins.

In patent EP0865464, the process comprises the steps of depolymerization of coloured polyester, for example green PET, in the presence of a diol, in particular ethylene glycol, in a reactor at a temperature of between 180° C. and 240° C., optionally of evaporation in a thin-film evaporator, of dissolution in a hot solvent and a step of filtration to separate off insoluble impurities with a size of greater than 50 μm. The low proportion of pigments in coloured PET enables separation by filtration. However, this technology cannot operate with a larger amount of pigments, for example present in opaque PET, since these pigments rapidly clog the filter.

Patent JP3715812 describes the production of refined BHET from PET. The process comprises: the glycolysis of the PET flakes, pretreated beforehand by washing with water in solid form, in the presence of ethylene glycol and a catalyst in a stirred reactor at 180° C., and then at 195-200° C.; followed by a step of prepurification by cooling, filtration, adsorption and treatment on ion-exchange resin. This prepurification step is presented as being important and being carried out before the evaporation of the glycol and the purification of the BHET for preventing the re-polymerization of the BHET in the subsequent purification steps. However, passing through a filtration and ion-exchange resin step can be extremely problematic when the feedstock comprises a large amount of very small solid particles such as pigments.

Lastly, patent application FR 3053691 describes a process for depolymerization of a polyester feedstock comprising opaque PET and in particular from 0.1% to 10% by weight of pigments, by glycolysis in the presence of ethylene glycol. A purified bis(2-hydroxyethyl) terephthalate (BHET) effluent is obtained after particular steps of separation and of purification by adsorption. However, the BHET effluent obtained by the depolymerization process described in patent application FR 3053691 may have imperfections: the BHET effluent obtained notably undergoes rapid colouration, despite being passed through a column of adsorbent.

The present invention seeks to improve the purification of a crude diester monomer, and in particular the purification step of the prior art processes, such as those mentioned above, in order to improve the decolourization of a diester monomer, in particular of the BHET monomer, notably obtained after depolymerization of a polyester feedstock comprising PET. The objective of the invention is specifically to obtain a diester monomer, in particular a BHET monomer, of high purity and decolourized, from a crude diester monomer feedstock, in particular derived from a reaction for depolymerization by glycolysis of polyester waste and in particular of PET waste.

### SUMMARY OF THE INVENTION

The aim of the invention is thus a process for purifying a crude diester monomer feedstock, the process comprising:
a) a mixing step fed by the crude diester monomer feedstock and an aqueous solvent, and carried out at a temperature of between 60° C. and 150° C., in order to obtain an aqueous mixture of diester monomer, the amount of aqueous solvent introduced being adjusted so that the crude diester monomer feedstock represents between 20% and 90% by weight of the total weight of the diester monomer aqueous mixture;
b) an adsorption step carried out by contacting the diester monomer aqueous mixture with at least one adsorbent, at a temperature of between 60° C. and 150° C. and at a pressure of between 0.1 and 1.0 MPa, in order to obtain a purified monomer effluent.

One advantage of the present invention lies in obtaining, from a crude diester monomer feedstock, in particular a crude BHET feedstock, a diester monomer effluent, in particular a BHET effluent, which is purified and decolourized. When it is in liquid form, the purified diester monomer effluent, obtained at the end of the process according to the invention, is advantageously colourless or near colourless, to the eye; when it is treated in order to obtain an effluent in solid form, the purified diester monomer effluent in solid form is a white solid, to the eye. Advantageously, the process according to the invention makes it possible to obtain a purified diester monomer effluent that does not exhibit any significant absorption band (that is to say indistinguishable from background noise) within the range of visible wavelengths, that is to say between 400 and 800 nm, when it is characterized by UV-visible spectrometry. Very advantageously, the purified diester monomer effluent, obtained at the end of the process according to the invention, has, preferably in solid form, colour parameters expressed in the CIE 1976 L*a*b* reference system, determined by colourimetry (according to the ASTM D6290 2019 method), preferably with:
a lightness (or luminance) parameter L* of close to 100, more particularly greater than 90.00 and preferably greater than 92.00 (100.00 being the maximum);
a parameter a* (corresponding to a green-red axis) of close to 0, more particularly between −1.50 and +1.50 and preferably between −1.00 and +1.00; and
a parameter b* (corresponding to a blue-yellow axis) of close to 0, more particularly between −2.50 and +2.50, more particularly between −1.00 and +1.50.

An advantage of the invention is therefore to obtain a purified and decolourized diester monomer effluent from a crude diester monomer stream, derived in particular from a process for depolymerization by glycolysis of polyester waste which typically comprises coloured, opaque or even multilayer PETs and therefore consequently pigments and dyes. The process according to the invention thus makes it possible to remove the residual impurities, such as dyes and/or organic or inorganic salts, which might not have been eliminated during separation steps downstream of the depolymerization of the polyester.

Such a diester monomer may then be subsequently (re)polymerized to give a polyester polymer which exhibits no difference from a virgin polyester, in particular a virgin PET, thus allowing access to all of the uses of virgin PET.

The process according to the invention is very flexible and can easily be incorporated downstream of any process for depolymerization, in particular by glycolysis, of polyester, such as PET and including opaque and/or coloured PET, as a step for purifying the diester monomer effluent obtained directly by the depolymerization reaction or after steps for separating the diol introduced in excess for the glycolysis or generated during the depolymerization and/or heavy impurities such as incompletely converted oligomers and pigments. For example, the process according to the invention may easily be incorporated in place of the decolourization step of the process described in patent application FR 3053691.

### LIST OF FIGURES

FIG. 1 shows the UV-visible spectra obtained between 550 nm and 700 nm, for the effluents produced by the processes described in Examples 1, 2 and 3.

FIG. 2 shows the UV-visible spectrum obtained between 350 nm and 850 nm, for the effluent produced by the process described in Example 1.

### DESCRIPTION OF THE EMBODIMENTS

According to the invention, polyethylene terephthalate or poly(ethylene terephthalate), also simply called PET, has an elementary repeating unit comprising a terephthalic acid diester, of formula:

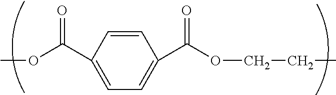

Conventionally, PET is obtained by polycondensation of terephthalic acid (PTA) or dimethyl terephthalate (DMT) with ethylene glycol.

According to the invention, the term “monomer” or “diester monomer” advantageously denotes the repeating unit of a polyester polymer, and defines a diester of a dicarboxylic acid, preferably of an aromatic dicarboxylic acid and preferentially of terephthalic acid, and of a diol comprising preferably between 2 and 12 carbon atoms, preferentially between 2 and 4 carbon atoms, the preferred diol being ethylene glycol. More particularly, the “monomer” or “diester monomer” corresponds to the product targeted by the process according to the invention. Thus, according to one embodiment of the invention, the “monomer” or “diester monomer”, the product targeted by the invention, has a chemical formula of the type: HOCₙH₂ₙ—CO₂-(Aro)-CO₂—CₙH₂ₙOH, with n=2-12, preferably n=2-4 and -(Aro)-=—(C₆H₄)— representing an aromatic ring. Preferably, the term “monomer” or “diester monomer” denotes bis(2-hydroxyethyl) terephthalate (BHET) of chemical formula HOC₂H₄—CO₂—(C₆H₄)—CO₂—C₂H₄OH, in which —(C₆H₄)— represents an aromatic ring.

The term “oligomer” typically denotes a polymer of small size, consisting generally of 2 to 20 elementary repeating units. According to the invention, the term “ester oligomer” or “BHET oligomer” denotes a terephthalate ester oligomer comprising between 2 and 20, preferably between 2 and 5, elementary repeating units of formula —[O—CO—(C₆H₄)—CO—O—C₂H₄]—, where —(C₆H₄)— is an aromatic ring.

According to the invention, the terms “diol” and “glycol” are used without distinction and correspond to compounds comprising 2 hydroxyl —OH groups and preferably comprising between 2 and 12 carbon atoms, preferentially between 2 and 4 carbon atoms. The preferred diol is ethylene glycol, also referred to as monoethylene glycol or MEG.

Therefore, the diol or diol effluent streams optionally used in the steps of the process of the invention thus preferably comprise ethylene glycol (or MEG) in an amount of greater than 40% by weight, preferentially greater than 50% by weight, preferably greater than or equal to 60% by weight, of the total weight of said diol or diol effluent stream.

The term “dye” defines a substance that is soluble in the polyester material and that is used to colour it. The dye may be of natural or synthetic origin.

According to the invention, the term “pigment”, more particularly opacifying and/or colouring pigment, defines a finely divided substance which is insoluble in particular in the polyester material. The pigments are in the form of solid particles with a size of generally between 0.1 and 10 μm, and predominantly between 0.4 and 0.8 μm. They are often of inorganic nature. The pigments generally used, in particular for opacifying, are metal oxides, such as TiO₂, CoAl₂O₄ or Fe₂O₃, silicates, polysulfides and carbon black.

According to the present invention, the expressions “of between . . . and . . . ” and “between . . . and . . . ” are equivalent and mean that the limiting values of the interval are included in the range of values which is described. If such were not the case and if the limit values were not included in the described range, such a clarification will be given by the present invention.

For the purposes of the present invention, the various ranges of parameters for a given step, such as the pressure ranges and the temperature ranges, may be used alone or in combination. For example, within the meaning of the present invention, a range of preferred pressure values can be combined with a range of more preferred temperature values.

In the text hereinbelow, particular embodiments of the invention may be described. They may be implemented separately or combined together without limitation of combinations when this is technically feasible.

The terms “upstream” and “downstream” should be understood as a function of the general flow of the stream in the process.

According to the present invention, the pressures are absolute pressures and are given in MPa or MPa absolute (or MPa abs).

### Feedstock

The process according to the invention is fed by a crude diester monomer feedstock.

Said crude diester monomer feedstock advantageously comprises at least 70.0% by weight, preferably at least 85.0% by weight, preferentially at least 95.0% by weight, preferably at least 99.0% by weight and very preferably at least 99.9% by weight, of a diester monomer, preferably of bis(2-hydroxyethyl) terephthalate (or BHET), 100.0% by weight representing the maximum of diester monomer in the feedstock. The crude diester monomer feedstock may also comprise impurities, preferably which are soluble in the diester monomer. These impurities may also be referred to as residual impurities. These are for example dyes, typically used to colour the polyester polymer material, organic or inorganic salts, or compounds of the type of esters of a dicarboxylic acid, preferably of an aromatic dicarboxylic acid and preferentially of terephthalic acid, and of at least one diol dimer or trimer (said diol being the one forming the targeted diester monomer) preferably comprising between 4 and 36 carbon atoms, preferentially between 4 and 8 carbon atoms, such as for example diethylene glycol (one such impurity is for example 2-(2-hydroxyethoxy)ethyl 2-hydroxyethyl terephthalate, which is an ester of terephthalic acid with ethylene glycol and diethylene glycol). More particularly, the impurities of dye type may represent up to 1% by weight (i.e. less than 1% by weight), preferably up to 0.1% by weight, preferentially up to 0.05% by weight, of the total weight of the crude diester monomer feedstock, and in particular at least 1 ppm by weight of the total weight of the crude diester monomer feedstock. The other impurities, in particular of the type of esters of dicarboxylic acid and at least one diol dimer or trimer, may represent in total up to 15.0% by weight, preferably up to 10.0% by weight, preferentially up to 5.0% by weight, of the total weight of the crude diester monomer feedstock, and in particular at least 10 ppm by weight of the total weight of the crude diester monomer feedstock.

The crude diester monomer feedstock may optionally further comprise a solvent such as a monoalcohol, in particular methanol or ethanol, or a diol, more particularly ethylene glycol. The crude diester monomer feedstock may in particular comprise up to 30.0% by weight, preferably up to 15.0% by weight, preferentially up to 5.0% by weight, preferably up to 1.0% by weight and very preferably up to 0.1% by weight of solvent, and more particularly of diol such as ethylene glycol, or only traces of solvent and in particular less than 500 ppm by weight of solvent, preferably of diol and in particular of ethylene glycol. The crude diester monomer feedstock may also not comprise any solvent.

According to one preferred embodiment of the invention, the crude diester monomer feedstock is derived from a process for depolymerization by glycolysis of a polyester feedstock, in particular of a polyester waste feedstock which typically comprises coloured and/or opaque PETs, and optionally multilayer PETs. More particularly, the crude diester monomer feedstock may be derived directly or indirectly from the depolymerization, in the presence of a diol, preferably ethylene glycol, of a polyester feedstock comprising coloured and/or opaque PET, the term “indirectly derived” meaning that the depolymerization process comprises steps of prepurification of the reaction effluent obtained by the depolymerization reaction in the presence of diol such as for example a step of separating the diol used in excess for the glycolysis or generated during the depolymerization and/or a step of separating the heavy impurities such as the incompletely converted oligomers and/or a step of separating the ionic species for example by passing through ion-exchange resins, the term “directly derived” meaning that the depolymerization process does not comprise such prepurification steps.

According to one very particular embodiment of the invention, the crude diester monomer feedstock which feeds the purification process according to the invention is derived from a depolymerization process such as the one described in patent application FR 3053691 and in which the purification process according to the invention replaces the decolourization step described.

### Purification Process

The purification of the crude diester monomer feedstock is advantageously carried out by adsorption of an aqueous solution of crude diester monomer.

The process according to the invention thus comprises at least a step a) for mixing the diester monomer feedstock with an aqueous solvent, an adsorption step b) by contacting the obtained diester monomer aqueous mixture with at least one adsorbent to obtain a purified diester monomer effluent.

The process according to the invention may optionally comprise a step c) for separating the diester monomer in order to obtain a separated purified diester monomer effluent and a spent aqueous solvent effluent. The process according to the invention may also optionally comprise an additional step c*) for crystallizing the diester monomer, preferably located downstream of the adsorption step b) and advantageously upstream of an optional separation step c).

### Mixing Step a)

Advantageously, the mixing step a) is fed by the crude diester monomer feedstock, which preferably comprises BHET, and an aqueous solvent. Step a) makes it possible to obtain a diester monomer aqueous mixture.

The aqueous solvent advantageously comprises water, and preferably at least 50% by weight of water, preferably at least 75% by weight of water, preferentially at least 90% by weight of water, more preferentially still at least 97% by weight of water, and preferably at least 99% by weight of water, the maximum being 100% by weight of water (that is to say that the aqueous solvent advantageously comprises 100% by weight of water or less, and in particular between 50% and 100% by weight, preferably between 75% and 100% by weight, preferentially between 90% and 100% by weight, more preferentially between 97% and 100% by weight, and preferably between 99% and 100% by weight of water). Optionally, the aqueous solvent, besides water, may comprise other water-miscible compounds, for example of alcohol, diol, acid, etc. type. The aqueous solvent may also comprise ions and/or mineral salts in small amounts, typically in an amount of less than 1% by weight. Preferably, the aqueous solvent comprises at least 99% by weight of water, and in particular up to 100% by weight of water, and may optionally comprise ions and/or mineral salts. According to a preferred embodiment of the invention, the aqueous solvent that feeds the mixing step a) comprises, preferably consists of, all or part of an aqueous solvent effluent, that is optionally purified, derived from a spent aqueous solvent effluent obtained at the outlet of an optional separation step c), and optionally supplemented by a supply of solvent external to the process according to the invention.

Preferably, the amount of aqueous solvent introduced is adjusted so that the crude diester monomer feedstock represents between 20% and 90% by weight, preferentially between 30% and 80% by weight, preferably between 40% and 75% by weight and more preferably still between 40% and 60% by weight, of the total weight of the diester monomer aqueous mixture.

Advantageously, the mixing step a) is carried out at a temperature of between 60° C. and 150° C., preferably between 70° C. and 120° C. and preferably between 75° C. and 110° C., and preferably at a pressure of between 0.1 and 1.0 MPa, preferably between 0.1 and 0.8 MPa, and with preference between 0.1 and 0.5 MPa.

The aqueous solvent may be heated, prior to said mixing step a), preferably at the temperature at which the mixing step a) is carried out, in particular at a temperature of between 60° C. and 150° C., preferably between 70° C. and 120° C. and preferably between 75° C. and 110° C. Preferably, the crude diester monomer feedstock feeds the mixing step a) at a temperature at which said crude diester monomer feedstock is at least partly, preferably completely, in liquid or molten form. Very advantageously, the crude diester monomer feedstock may be heated, prior to the mixing step a), preferably at a temperature greater than or equal to 110° C., preferably greater than or equal to 120° C., and preferably less than or equal to 220° C., preferentially less than or equal to 200° C. Thus, the crude diester monomer feedstock, which preferably comprises BHET, advantageously feeds the mixing step a) at a temperature (or inlet temperature) greater than or equal to 110° C., preferably greater than or equal to 120° C., and preferably less than or equal to 220° C., preferentially less than or equal to 200° C.

The mixing step a) may use any mixing equipment known to those skilled in the art, such as for example a static or dynamic mixer, in particular a static mixer.

The diester monomer aqueous mixture obtained at the end of step a) is advantageously a homogeneous mixture in which the diester monomer, in particular BHET, is soluble.

### Adsorption Step b)

The diester monomer aqueous mixture obtained at the end of step a) feeds an adsorption step b). The adsorption step b) is carried out by contacting the diester monomer aqueous mixture with at least one adsorbent, in particular which is solid, advantageously at a temperature of between 60° C. and 150° C., preferably between 70° C. and 120° C., preferentially between 75° C. and 110° C., and very advantageously at a pressure of between 0.1 and 1.0 MPa, in particular between 0.1 and 0.8 MPa, and more particularly between 0.1 and 0.5 MPa.

The adsorption step b) advantageously uses at least one adsorption unit (also referred to as adsorption train), preferably between one and ten adsorption units, preferably between one and four adsorption units, each adsorption unit advantageously operating in parallel relative to one another. Advantageously, each adsorption section comprises at least one adsorber and preferably up to four adsorbers, each adsorber being for example a reactor or a column. Very advantageously, the residence time in each adsorber of the adsorption step is preferably between 20 minutes and 40 hours, preferably between 1 hour and 30 hours, preferably between 1 hour and 20 hours. The residence time is defined here as the ratio between the internal volume of the adsorber in question and the volume flow rate of the diester monomer aqueous mixture from the mixing step a).

The adsorption step b) uses at least one adsorbent, in particular which is solid, and preferably up to five different adsorbents. According to a very particular embodiment, the adsorption step b) uses one or two different adsorbents. According to the invention, adsorbents are said to be different when their nature and/or their composition and/or their particle size and/or their textural characteristics, such as the pore volume, is/are different. Preferably, the different adsorbents are of different nature. Specifically it may be advantageous to combine several different adsorbents, in particular of different nature, in order to optimize the removal of the residual impurities, in particular of the residual dyes or of the residual salts, which may themselves be of very different nature. Specifically, polyester waste, such as PET packaging or plastic bottle waste, from which the crude diester monomer feedstock treated by the process according to the invention may be obtained, by depolymerization of said waste, may comprise a very large number of coloured and/or opaque PETs and therefore a very large number of different dye compounds. The colouring of the crude diester monomer feedstock may also originate from a degradation or transformation of compounds contained in the polyester waste during various steps of a depolymerization process from which the crude diester monomer feedstock may be derived (for example the waste conditioning steps and/or depolymerization reaction steps).

When the adsorption step b) uses between two and five different adsorbents, said different adsorbents are in a mixture or are placed in series in one adsorber or several adsorbers, advantageously in one and the same adsorption unit. Preferably, when the adsorption step b) uses between two and five different adsorbents, said different adsorbents are in series relative to one another, advantageously in one and the same adsorption unit, and more preferentially each of the adsorbents are in different adsorbers placed in series or in parallel, preferably in series, advantageously in one and the same adsorption unit.

Advantageously, the adsorbent(s), which is/are in particular solid, is/are chosen from activated carbons, alumina and clays. Activated carbons are for example obtained from petcoke, from bituminous coal or from any other fossil origin, or obtained from biomass such as wood, coconut or any other source of biomass. Various raw materials may also be mixed in order to obtain activated carbons which may be used as adsorbents in said adsorption step b). The clays may be layered double hydroxides or natural or converted clays such as those known to a person skilled in the art as decolourizing earths. Preferably, at least one adsorbent is an activated carbon. Thus, when the adsorption step b) uses a single type of adsorbent, said adsorbent is an activated carbon and, when the adsorption step b) uses two or more different adsorbents, one adsorbent is an activated carbon and the other(s) is/are another activated carbon, an alumina and/or a clay, preferably an activated carbon and/or a clay, more particularly a clay.

Preferably, each adsorbent used in the adsorption step b) has a pore volume (Vp), determined by mercury porosimetry, of greater than or equal to 0.25 ml/g, preferentially greater than or equal to 0.40 ml/g, more preferably greater than or equal to 0.50 ml/g, and preferably less than or equal to 5 ml/g.

Preferably, the adsorption step b) is carried out, advantageously in each adsorption unit:
in flow-through fixed-bed (or fixed-bed) mode, that is to say in at least one adsorber comprising a fixed bed of adsorbent(s), in particular at least one column of adsorbent(s), which may operate in upflow or downflow mode, preferably in upflow mode, or
in stirred mode, advantageously in at least one continuous stirred reactor, also known as continuous stirred tank reactor (CSTR).

In the case where the adsorption step b) is carried out in stirred mode in at least one CSTR-type stirred reactor, the reactor(s) is/are followed by a filtration system for recovering said adsorbent(s) which is/are in suspension in the liquid treated. Preferably, the adsorption step b) is carried out in flow-through fixed-bed mode, advantageously in each adsorption unit.

Preferably, in the case where the adsorption step b) uses, advantageously in each adsorption unit, at least two different adsorbents, in flow-through fixed-bed mode, the adsorbents may be:
all present in each adsorber, or column, used, as a mixture or in successive fixed beds, or
each used in an adsorption section, the sections being placed in series relative to one another, each adsorption section consisting of between one and four, preferably between two and four, fixed-bed adsorbent columns, advantageously in each adsorption unit.

Very advantageously, the adsorption step b), advantageously each adsorption unit or each of the adsorption sections, uses several fixed-bed columns, in particular at least two fixed-bed columns, preferably between two and four fixed-bed columns, of the same adsorbent(s). When the adsorption step b), advantageously in each adsorption unit or one adsorption section, uses two columns of the same adsorbent(s), the adsorption step b), advantageously in each adsorption unit or the adsorption section, may operate according to a “swing” operating mode in which one of the columns is on-line while the other column is in reserve. When the adsorbent in the on-line column is spent, this column is isolated, while the column in reserve is placed on-line. The spent adsorbent of the isolated column may then be regenerated in situ and/or replaced with fresh adsorbent, to be placed back on-line again once the other column has been isolated. Another operating mode of the adsorbent columns is to have at least two columns operating in series, advantageously in each adsorption unit: when the adsorbent of the lead column (that is, the first column in the series) is spent, this first column is isolated and the spent adsorbent is regenerated in situ or replaced with fresh adsorbent, said column then being placed back on-line in the last position of the series of columns and so on. This operation is referred to as “lead-lag”. Very preferably, the adsorption step b), advantageously in each adsorption unit or each adsorption section, uses at least two columns of the same adsorbent, preferably two to four columns of the same adsorbent, preferentially two columns of the same adsorbent, operating in “lead-lag” mode.

The combination of at least two columns of the same adsorbent, advantageously in each adsorption unit, makes it possible in particular to remedy the, possibly rapid, saturation and/or clogging of the adsorbent. Specifically, the presence of at least two columns of adsorbent facilitates the replacement and/or the regeneration of the adsorbent, advantageously without shutting down the adsorption unit, or even the process, thus making it possible to reduce the risks of clogging, prevent shutdowns of the unit due to saturation of the adsorbent, to manage costs and to limit the consumption of adsorbent, while ensuring continuous production of purified diester monomers. This combination of at least two columns of adsorbent, in particular operating in “lead-lag” mode, advantageously in each adsorption unit, also makes it possible to maximize the adsorption capacity of said adsorbent.

In a very particular embodiment of the invention, in which the adsorption step b) uses two different adsorbents, advantageously in each adsorption unit, the adsorption step b) very preferentially comprises a first adsorption section comprising at least two, preferably between 2 and 4, fixed-bed columns of activated carbon, operating in swing or in lead-lag mode, and a second adsorption section comprising at least two, preferably between 2 and 4, fixed-bed columns of another adsorbent, preferably chosen from another activated carbon or a clay, operating in particular in swing or in lead-lag mode, and placed upstream or downstream of the first section of fixed-bed activated carbon columns.

Each adsorbent used in the adsorption step b) is preferably in the form of granules, extrudates or powder. Preferably, each adsorbent is:
in the form of granules or extrudates when the adsorption step b) is carried out in flow-through fixed-bed mode, and
in the form of powder when the adsorption step b) is carried out in a CSTR-type stirred reactor.

The size of said at least one adsorbent, in particular when it is in the form of granules or extrudates, is such that the smallest dimension of said at least one adsorbent (corresponding to the diameter of the circle circumscribed on the basis of the granules or polylobal extrudates or to the diameter of the cylinder circumscribed on the cylindrical basis of the extrudates of cylindrical type; this dimension also being called “diameter”) is preferably between 0.1 and 5 mm, preferentially between 0.3 and 2 mm. For example, the activated carbon extrudates 0.8 mm in diameter sold by Cabot Norit or the granules within the size range between 0.4 and 1.7 mm sold by Chemviron may be suitable as adsorbent.

The process according to the invention may advantageously also comprise a phase of regenerating said adsorbent(s) of the adsorption step b).

A purified monomer effluent is obtained at the end of the adsorption step b). It may feed an optional separation step c) or optionally a crystallization step c*).

### Optional Crystallization Step c*)

The process according to the invention may optionally comprise a step c*) for crystallizing the diester monomer, preferably located downstream of the adsorption step b) and very advantageously upstream of an optional separation step c). Advantageously, the crystallization step c*) uses at least one solids production section. The optional crystallization step c*) makes it possible to obtain an aqueous suspension of solid diester monomer.

This optional crystallization step has a two-fold effect: it makes it possible to facilitate the separation of the purified diester monomer in solid form, from the spent aqueous solvent effluent, and also to improve the purification of the diester monomer.

According to one preferred embodiment of the invention, the process comprises a crystallization step c*), downstream of the adsorption step b) and very advantageously upstream of an optional separation step c). According to another embodiment of the invention, the process may comprise, downstream of the adsorption step b), between two and four crystallization steps c*), each step being followed by an optional separation step c) as described below.

According to another embodiment of the invention, the process may comprise at least one crystallization step c*) upstream of the adsorption step b) and advantageously upstream of the mixing step a). In the latter embodiment, the crystallization step c*) comprises a solids production section, fed by the optionally filtered, crude diester monomer feedstock, and a crystallization solvent as described below, and a solid-liquid separation section in order to separate the solid formed in the solids production section, the separated solid then being sent to the mixing step a) in which it will be dissolved in the aqueous solvent.

The optional crystallization step c*), and more particularly the solids production section, advantageously located downstream of the adsorption step b), is fed by the purified monomer effluent from the adsorption step b). The crystallization step c*) may optionally further comprise a section for filtering the purified monomer effluent from the adsorption step b), which section is located upstream of the solids production section.

Optionally, the solids production section may also be fed with a crystallization solvent which is identical to or different from the aqueous solvent introduced into the mixing step a). The crystallization solvent is advantageously chosen from: water; aqueous solvents comprising at least 50% by weight, preferably at least 75% by weight, preferentially at least 90% by weight, more preferentially still at least 97% by weight and preferably at least 99% by weight of water; monoalcohols preferably having between 1 and 12 carbon atoms, such as methanol or ethanol; diols preferably having between 1 and 12 carbon atoms; ethers; aldehydes; esters; hydrocarbons, preferably aromatic hydrocarbons, for example monoaromatic compounds; and the mixtures of at least two of these compounds belonging to the same chemical family or different chemical families. Preferably, the crystallization solvent is chosen from: water; an aqueous solvent comprising at least 50% by weight, preferably at least 75% by weight, preferentially at least 90% by weight, more preferentially still at least 97% by weight and preferably at least 99% by weight of water; a monoalcohol having between 1 and 12 carbon atoms, such as methanol or ethanol; a diol having between 1 and 12 carbon atoms, preferably ethylene glycol; a monoaromatic compound, for example xylene; and a mixture thereof. Preferably, the crystallization solvent is the same as the aqueous solvent introduced into the mixing step a).

According to a preferred embodiment of the invention, the crystallization solvent comprises, preferably consists of, all or part of an aqueous solvent effluent, which is optionally purified, derived from the spent aqueous solvent effluent obtained at the outlet of an optional separation step c), optionally supplemented by a supply of solvent external to the process according to the invention.

Preferably, when a crystallization solvent is introduced, the amount of crystallization solvent introduced into the solids production section is adjusted so that the crude diester monomer feedstock which feeds the process, and in particular the mixing step a) of the process, represents between 1% and 75% by weight, preferentially between 5% and 45% by weight, preferably between 15% and 35% by weight, of the total weight of the mixture in said solids production section (i.e. the mixture comprising the crude diester monomer feedstock, the aqueous solvent introduced into the mixing step a) and the crystallization solvent).

Prior to said solids production section, all or part of the crystallization solvent may be heated, preferably to the temperature at which the adsorption step b) is carried out, or cooled and in particular brought to a temperature preferably of between 0° C. and 120° C., preferably between 5° C. and 100° C., and preferably between 10° C. and 90° C.

Advantageously, the solids production section of the optional crystallization step c*) is operated at a temperature of (i.e. such that the temperature of the effluent from said solids production section is) between 0° C. and 100° C., preferably between 5° C. and 80° C., and preferably between 10° C. and 70° C. More precisely, in the solids production section, the purified monomer effluent from the adsorption step b) optionally in a mixture with the crystallization solvent, is cooled from the temperature at which the adsorption step b) is carried out, i.e. from a temperature of between 60° C. and 150° C., preferably between 70° C. and 120° C., preferentially between 75° C. and 110° C., to a temperature of between 0° C. and 100° C., preferably between 5° C. and 80° C., and preferably between 10° C. and 70° C.

The cooling may be implemented according to any method known to a person skilled in the art. For example, in batch mode, the cooling of the temperature may be realized without regulation of the lowering of the temperature (i.e. without an imposed temperature ramp; thus only the initial and final temperatures are controlled) or according to at least one decreasing temperature ramp, in particular according to a decreasing temperature ramp of between 5° C. and 30° C./hour and more particularly between 8° C. and 15° C./hour, or else according to both modes chained together in succession, i.e. without control for one part of the cooling and according to a decreasing temperature ramp for another part of the cooling. According to another example, the cooling may simply be due to the introduction of the stream to be cooled, i.e. the purified monomer effluent from the adsorption step b) or the mixture comprising the purified monomer effluent and the crystallization solvent, into a reservoir having a volume which is advantageously adapted to the flow rate of the stream to be cooled and which is held at a temperature of between 0° C. and 100° C., preferably between 5° C. and 80° C., and preferably between 10° C. and 70° C.

The solids production section is advantageously operated at a pressure of between 0.00001 and 1.00 MPa, preferably between 0.0001 and 0.50 MPa, and with preference between 0.001 and 0.20 MPa. According to a particular embodiment of the invention, the solids production section is operated under vacuum, preferably at a pressure of between 0.0001 and 0.10 MPa, preferentially between 0.001 and 0.01 MPa. According to another particular embodiment, the solids production section is advantageously operated in a jacketed reactor, at a pressure of between 0.01 and 1.00 MPa, preferably between 0.05 and 0.20 MPa, preferably at atmospheric pressure, i.e. at 0.10 MPa.

According to a preferred embodiment of the invention, water, as crystallization solvent, is mixed with the purified monomer effluent from step b) and the solids production section is operated under conditions such that the temperature of the effluent from said solids production section is between 5° C. and 50° C., preferably between 10° C. and 40° C.

According to another preferred embodiment of the invention, the crystallization solvent introduced and mixed with the purified monomer effluent from step b) is ethylene glycol and the solids production section is operated under conditions such that the temperature of the effluent from said solids production section is between 5° C. and 50° C., preferably between 10° C. and 40° C.

Advantageously, the objective of the solids production section is to solidify, i.e. crystallize or precipitate, at least partly, the diester monomer, preferably the BHET. Thus, the solids production section comprises, and preferably consists of, a precipitation or crystallization phase carried out by any precipitation or crystallization technique known to a person skilled in the art. The solids production section is preferably a section for crystallization, for example by cooling or by concentration, which is carried out in any equipment known to a person skilled in the art, as defined, for example, in the journal Techniques de l'Ingénieur, “Cristallisation industrielle—Aspects pratiques” [Industrial Crystallization—Practical Aspects], ref. J2788 V1, followed by a liquid-solid separation.

Advantageously, the section for producing solids, preferably by crystallization, comprises one or more crystallization operations, operating in series or in parallel, which is/are carried out batchwise or continuously, preferably continuously.

The solids production section of the optional crystallization step c*) makes it possible to obtain a heterogeneous effluent, and more particularly an aqueous suspension of solid diester monomer, comprising a solid phase of diester monomer and a liquid phase which may comprise possible residual impurities, such as residual dyes, still present in the purified monomer effluent resulting from the adsorption step b). Advantageously, the aqueous suspension of solid diester monomer obtained at the end of an optional crystallization step c*) is sent to a separation step c).

### Optional Separation Step c)

The purification process may comprise, preferably comprises, a separation step c) located downstream of step b). The optional separation step is advantageously fed by the purified monomer effluent from the adsorption step b) or by the aqueous suspension of solid diester monomer obtained at the end of the optional crystallization step c*) when the process according to the invention comprises such a step.

The separation step c), when it is incorporated into the process according to the invention, advantageously makes it possible to separate a purified diester monomer effluent and a spent aqueous solvent effluent.

The optional separation step c) may advantageously use any separation technique known to those skilled in the art. The optional separation step c) may for example use a separation by distillation and/or evaporation of the aqueous solvent, in order to obtain, on the one hand, the separated purified diester monomer effluent and, on the other hand, the spent aqueous solvent effluent which comprises the aqueous solvent. According to another embodiment, and in particular when the process according to the invention comprises a crystallization step c*), in particular downstream of the adsorption step b), the optional separation step c) may use a solid-liquid separation, such as for example a separation by filtration, by decantation and/or centrifugation, in order to separate the solid diester monomer advantageously in the form of crystals, and preferably BHET crystals, from the liquid phase of the aqueous suspension of solid diester monomer obtained at the end of the crystallization step c*). The solid diester monomer thus separated constitutes the separated purified diester monomer effluent, the liquid phase constituting the spent aqueous solvent effluent.

Very advantageously, the temperature and the pressure in the optional separation step c) are adjusted by a person skilled in the art in order to satisfactorily separate a purified diester monomer effluent and a spent aqueous solvent effluent. According to the embodiment in which the optional separation step c) uses a solid-liquid separation, the temperature at which step c) is carried out varies between 0° C. and 100° C., preferably between 5° C. and 80° C., and preferably between 10° C. and 50° C., and the pressure preferably varies between 0.0001 and 0.50 MPa, and preferably between 0.001 and 0.20 MPa.

According to a particular embodiment of the invention, the separated purified diester monomer effluent, recovered in solid form preferably by filtration or centrifugation, may additionally advantageously undergo all or some of the following operations, carried out one or more times without a predefined chronological order: rinsing with a solvent, identical to or different from the solvent feeding the mixing section or optionally the solids production section; additional filtration or centrifugation; removal of the residual solvent by any method known to a person skilled in the art, for example by evaporative drying; shaping, for example into powder or granules; and storage of the solid.

According to another embodiment of the invention, the separated purified diester monomer effluent is recovered, preferably by filtration or centrifugation, then is directly sent (i.e. without a phase of storage of the solid) to a polymerization step known to a person skilled in the art, optionally with, prior to the polymerization reaction, an operation of rinsing with water or a diol effluent, such as ethylene glycol effluent, preferably an operation of rinsing with water, of the solid purified diester monomer effluent, followed by heating of the rinsed solid for the purposes of melting.

Very advantageously, the purified diester monomer effluent or the separated purified diester monomer effluent, obtained at the end of the process according to the invention, is: colourless or near colourless to the naked eye when it is in liquid form; white when it is in solid form. The purification process according to the invention which comprises a step b) of adsorbing a diester monomer aqueous solution, and optionally a step c*) of crystallizing said diester monomer, thus makes it possible to satisfactorily purify and decolourize the crude diester monomer feedstock, even if the latter is derived from a process for the polymerization of a polyester feedstock which comprises a significant amount of coloured and/or opaque PET. Specifically, the impurities, such as dyes, present in the diester monomer feedstock remained trapped by the adsorbent, at least in part, and/or, for at least one other portion, dissolved in the aqueous solvent or the mixture of solvents introduced during the process and may also be found in an optionally separated, spent aqueous solvent effluent.

Very advantageously, the purified diester monomer effluent or the separated purified diester monomer effluent, obtained at the end of the process according to the invention, preferably comprises at least 90% by weight, preferentially at least 95% by weight, preferably at least 98% by weight, of diester monomer (i.e. of the product targeted by the process according to the invention), preferably BHET, on the basis of the dry weight (i.e. relative to the solids contained in said purified or separated diester monomer effluent). The purified diester monomer effluent or the separated purified diester monomer effluent, obtained at the end of the purification process according to the invention, may comprise very advantageously less than 5% by weight, preferably 1% by weight and preferentially less than 0.5% by weight, of impurities of the type of esters of dicarboxylic acid with at least one diol dimer or trimer, such as ester compounds derived from diethylene glycol, on the basis of the dry weight (i.e. relative to the solids of said effluent).

The purified diester monomer effluent or the separated purified diester monomer effluent, obtained at the end of the process, may be characterized by UV-visible spectrometry in order to identify the presence of absorption bands in the visible range, in particular between 400 and 800 nm. According to this characterization method, the purified diester monomer effluent or the separated purified diester monomer effluent is preferably characterized by UV-visible spectrometry, in particular between 400 and 800 nm, advantageously in liquid medium, that is to say advantageously after dilution or dissolution in a suitable solvent, preferably at between 0.1% and 10% by weight, at ambient temperature, using a conventional laboratory counter-top UV-visible spectrometer. Ethanol may be used as a suitable solvent, allowing dilution or dissolution of a sample of the purified diester monomer effluent or the separated purified diester monomer effluent. A cuvette with a conventional 1 cm or 1 inch optical path length may be used. Preferably, the UV-visible spectrum of the diester monomer effluent or the separated purified diester monomer effluent is determined with the aid of a solution of said diester monomer effluent prepared at 5% by weight in ethanol, and of a cuvette with a 1 inch optical path length. According to this method, the purified or separated diester monomer effluent obtained by the process according to the invention advantageously exhibits a spectrum that preferably does not display any significant absorption band (i.e. that is indistinguishable from background noise) within the range of visible wavelengths (400-800 nm), and in particular in the range between 550 and 650 nm. Indeed, surprisingly, the process according to the invention which comprises a step of adsorption in water makes it possible to effectively eliminate the blue dyes which absorb visible light typically between 550 and 650 nm.

The purified diester monomer effluent or the separated purified diester monomer effluent, obtained at the end of the process, may also be characterized according to a colourimetry method such as described in ASTM D6290 2019. The illuminant chosen is illuminant D65, measurements are carried out in reflection and in specular excluded mode, 10⁰ standard observer. The measurements are expressed in the CIE L*a*b* reference system. According to the colourimetry method, the diester monomer effluent or the separated purified diester monomer effluent, obtained by the process according to the invention, preferably in solid form, advantageously exhibits a CIE L*a*b* reference system with:
a lightness (or luminance) parameter L* of close to 100, more particularly greater than 90.00 and preferably greater than 92.00 (100.00 being the maximum);
a parameter a* (corresponding to a green-red axis) of close to 0, more particularly between −1.50 and +1.50 and preferably between −1.00 and +1.00; and
a parameter b* (corresponding to a blue-yellow axis) of close to 0, more particularly between −2.50 and +2.50, and more particularly between −1.00 and +1.50.

The spent aqueous solvent effluent obtained at the end of the optional step c) comprises all or part of the aqueous solvent introduced into the mixing step a) and of the crystallization solvent optionally introduced into the optional crystallization step c*). It may also comprise dyes and/or other residual impurities. Preferably, the spent aqueous solvent effluent comprises less than 20% by weight, preferentially less than 15% by weight, preferably less than 10% by weight and preferably less than 5% by weight, of diester monomer (i.e. of the targeted product), preferably of BHET monomer.

The spent aqueous solvent effluent may then be, at least in part, directly recycled to the mixing step a) of the process and/or the optional crystallization step c*). The spent aqueous solvent effluent may also be treated, at least in part, so as to, in particular, separate the dyes and/or impurities, for example by adsorption, and thus recover a purified aqueous solvent which is then recycled, at least in part, to the mixing step a) of the process and/or the optional crystallization step c*). The spent aqueous solvent effluent may also undergo, in addition to the separation of the dyes and/or impurities, an operation for separation of solvents, for example by distillation or decantation, when a crystallization solvent is introduced and the crystallization solvent is different from the solvent introduced into the mixing step a), in order to then obtain two separate solvents, one able to be recycled to the mixing step a) and the second able to be recycled to the solids production section of the crystallization step c*).

The purified diester monomer effluent or the separated purified diester monomer effluent, obtained at the end of the process according to the invention, may thus feed, directly or indirectly, a polymerization step known to a person skilled in the art for the purpose of producing a polyester polymer, preferably PET or a PET-based copolyester, which is indistinguishable from the corresponding virgin resin. Said polymerization step may also be fed, in addition to the purified diester monomer effluent or the separated purified diester monomer effluent, with ethylene glycol, terephthalic acid or dimethyl terephthalate, or any other monomer, in accordance with the targeted (co)polymer.

The following figures and examples illustrate the invention without limiting the scope thereof.

### EXAMPLES

In the following examples, the steps leading to the production of the crude BHET feedstock are identical and are described below.

A polyester feedstock comprising, in particular, 20% by weight of opaque PET is obtained from collection and sorting channels for treatment. 4 kg/h of flakes of said polyester feedstock comprising 20% by weight of opaque PET which contains 6.2% by weight of TiO₂ pigment, are brought to a temperature of 250° C. and then injected with 11.5 kg/h of ethylene glycol (MEG) into a first stirred reactor which is maintained at 250° C., and then into a second and a third stirred reactor, which are maintained at 220° C. The reactors are maintained at a pressure of 0.4 MPa. The residence time, defined as the ratio of the liquid volume in the reactor to the sum of the liquid volume flow rates entering the reactor, is set at 20 min in the first reactor and 2.1 h in the second and third reactors. At the outlet of the third reactor, the reaction effluent consists of 67.7% by weight of diol composed very predominantly of ethylene glycol (MEG) (i.e. comprising 95% by weight or more of MEG), 25.8% by weight of diester monomer composed very predominantly of bis(2-hydroxyethyl) terephthalate (BHET) (i.e. comprising 95% by weight or more of BHET), 0.32% by weight of TiO₂, and 6.1% by weight of heavy compounds including inter alia dimers and/or oligomers.

The diol present in the reaction effluent is separated by evaporation in a succession of two flash vessels at temperatures ranging from 180° C. to 120° C. and pressures from 0.04 MPa to 0.004 MPa, followed by a thin-film evaporator operated at 175° C. and 0.0005 MPa. At the end of this evaporation step, an MEG-rich stream of 10.46 kg/h and a BHET-rich liquid stream of 5.02 kg/h are recovered. The BHET-rich liquid stream, corresponding to the liquid monomer effluent, consists of 79.6% by weight of BHET diester monomer, 0.6% by weight of MEG and 1.0% by weight of TiO₂ and 18.8% by weight of heavy compounds including inter alia dimers of BHET.

The BHET-rich liquid stream is then injected to a short-path evaporator, also referred to as short-path distillation, which is operated at a pressure of 20 Pa. A hot oil at 215° C. enables the evaporation of the BHET, which is subsequently condensed in the short-path evaporator at 130° C. to give a liquid stream of BHET as distillate of the short-path evaporator, having a flow rate of 3.8 kg/h. The residence time in the short-path evaporator is 1 min. The liquid stream of BHET recovered at the outlet of the short-path evaporator corresponds to the crude BHET, which is the feedstock for the purification processes described in Examples 1, 2 and 3 below. It consists of 99% by weight of BHET diester monomer and is devoid of any trace of TiO₂. A heavy residue with a flow rate of 1.19 kg/h is recovered as a residue from the short-path evaporator and comprises 16.7% by weight of BHET diester monomer, 79.2% by weight of BHET oligomers and 4.1% by weight of TiO₂.

### Example 1—in Accordance

The crude BHET is compressed to 0.15 MPa and feeds a mixing section which is also fed by a stream of water. The water feed flow rate is adjusted so that the crude BHET represents 50% by weight of the mixture (crude BHET+water). Said mixing section is operated at 90° C., at a pressure of 0.15 MPa.

The resulting mixture then feeds an adsorption section consisting of two columns each filled with an adsorbent (i.e. with a fixed bed of adsorbent). The adsorption section is operated at 90° C., at a pressure of 0.15 MPa. One column is placed on stream (i.e. it is in operation), the other remaining in reserve. The adsorbent used for packing the two columns is an activated carbon consisting of cylindrical extrudates with a diameter of 0.8 mm, reference ROY 0.8 from Cabot Norit.

The residence time is fixed at 40 minutes, in one column. The empty column linear velocity is 2.4 cm/min.

The effluent composed of BHET at 50% by weight in the BHET-water mixture is collected at the outlet of the column over time.

A measurement by UV-visible spectrometry is carried out on a BHET solution prepared with a sample of the effluent obtained at 40 hours of operation then dissolved in ethanol, so as to achieve a concentration of BHET in the final solution of 5% by weight. The measurement by UV-visible spectrometry is performed using a Hach DR3900 laboratory counter-top UV-visible spectrometer in a cuvette with an optical path length of one inch.

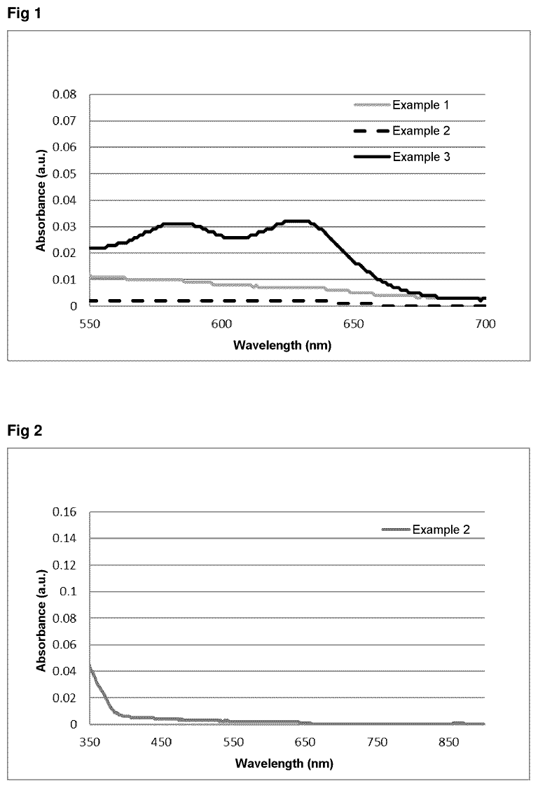

The spectrum obtained (cf. FIG. 1) does not display any significant absorption band in the wavelength range between 550 and 650 nm.

### Example 2—in Accordance

The crude BHET is compressed to 0.15 MPa and feeds a mixing section which is also fed by a stream of water. The water feed flow rate is adjusted so that the crude BHET represents 50% by weight of the mixture (crude BHET+water). Said mixing section is operated at 90° C., at a pressure of 0.15 MPa.

The resulting mixture then feeds an adsorption section consisting of two columns each filled with an adsorbent, in a fixed bed. The adsorption section is operated at 90° C., at a pressure of 0.15 MPa. One column is placed on stream (i.e. in operation), the other remaining in reserve. The adsorbent used for packing the two columns is an activated carbon consisting of cylindrical extrudates with a diameter of 0.8 mm, reference ROY 0.8 from Cabot Norit.

The residence time is fixed at 40 minutes, in one column. The empty column linear velocity is 2.4 cm/min.

780 g of the liquid stream from the adsorption step is mixed in a stirred tank with water, so as to achieve a final content of 20% by weight of BHET and 80% by weight of water, and a final temperature of 60° C. The mixture, kept under stirring, is cooled to 50° C. over 1 h and is then progressively cooled according to a ramp of 12° C./hour down to 20° C.

Solid particles form over the course of the cooling to afford a suspension of solids in a liquid comprising predominantly water. The suspension obtained at 20° C. is then filtered to recover a solid cake and a coloured liquid filtrate. The solid cake is rinsed with 1.5 l of water. The rinsed solid cake is recovered and then dried overnight under vacuum at 40° C. to afford 320 g of a white solid containing 99% by weight of BHET diester (determination of the composition by liquid chromatography).

The recovered solid is white. Measurement by UV-visible spectrometry is performed on a BHET solution prepared with a sample of the obtained white solid dissolved at 5% by weight in ethanol. The measurement by UV-visible spectrometry is performed using a Hach DR3900 laboratory counter-top UV-visible spectrometer in a cuvette with an optical path length of one inch.

The spectrum obtained (cf. FIG. 1 and FIG. 2) does not display any significant absorption band in the wavelength range between 400 and 800 nm.

Colourimetry measurements are also performed on the solid BHET obtained, in accordance with the method ASTM D6290 2019. A sample of 5 g of solid BHET product is ground in a mortar using a pestle. The 5 g of ground BHET are placed in a cuvette made of optical quality glass, with a diameter of 34 mm. The measurements are performed in reflection using a Konica Minolta CM-2300d colorimeter and SpectraMagic NX software, under the following conditions: illuminant D65, specular excluded, 10⁰ standard observer. The measurements are expressed in the CIE L*a*b* reference system. The result was obtained by averaging the values obtained for 10 measurements performed on the sample. The results are presented in Table 1.

TABLE 1

| | L* | a* | b* |
|---|---|---|---|
| Example 2 (in accordance) | 93.02 | 0.03 | 1.00 |

The colourimetry values obtained are in accordance with the targeted values.

### Example 3—not in Accordance

The crude BHET is compressed to 0.15 MPa and feeds a mixing section which is also fed by a stream of ethylene glycol. The ethylene glycol feed flow rate is adjusted so that the crude BHET represents 50% by weight of the mixture (crude BHET+ethylene glycol). Said mixing section is operated at 120° C., at a pressure of 0.15 MPa.

The resulting mixture then feeds an adsorption section consisting of two columns each filled with an adsorbent, in a fixed bed. The adsorption section is operated at 150° C., at a pressure of 0.15 MPa. One column is placed on stream (i.e. in operation), the other remaining in reserve.

The adsorbent used to fill the two columns is an activated carbon consisting of cylindrical extrudates with a diameter of 0.8 mm, reference ROY 0.8 from Cabot Norit.

The residence time is fixed at 40 minutes, in one column. The empty column linear velocity is 2.4 cm/min.

The effluent composed of BHET at 50% by weight in the BHET-ethylene glycol mixture is collected at the outlet of the column over time.

The product obtained at 40 hours of operation has a blueish colouration.

A measurement by UV-visible spectrometry is carried out on a BHET solution prepared with a sample of the effluent obtained at 40 hours of operation then dissolved in ethanol, so as to achieve a concentration of BHET in the final solution of 5% by weight. The measurement by UV-visible spectrometry is performed using a Hach DR3900 laboratory counter-top UV-visible spectrometer in a cuvette with an optical path length of one inch.

The spectrum obtained (cf. FIG. 1) has significant absorption bands in the wavelength range between 550 and 650 nm, consistent with the blueish colouration of the product.

1. Process for purifying a crude diester monomer feedstock, the process comprising:
a) a mixing step fed by the crude diester monomer feedstock and an aqueous solvent, and carried out at a temperature of between 60° C. and 150° C., in order to obtain a diester monomer aqueous mixture, the amount of aqueous solvent introduced being adjusted so that the crude diester monomer feedstock represents between 20% and 90% by weight of the total weight of the diester monomer aqueous mixture;
b) an adsorption step carried out by placing the crude diester monomer aqueous mixture in contact with at least one adsorbent, at a temperature of between 60° C. and 150° C. and at a pressure of between 0.1 and 1.0 MPa, in order to obtain a purified monomer effluent.

2. Process according to claim 1, wherein the aqueous solvent comprises at least 50% by weight of water, preferably at least 75% by weight of water, preferentially at least 90% by weight of water, more preferentially still at least 97% by weight of water, and preferably at least 99% by weight of water.

3. Process according to claim 1, wherein the amount of aqueous solvent introduced in step a) is adjusted so that the crude diester monomer feedstock represents between 30% and 80% by weight, preferably between 50% and 75% by weight and preferably between 40% and 60% by weight, of the total weight of the diester monomer aqueous mixture.

4. Process according to claim 1, wherein the mixing step a) is carried out at a temperature of between 70° C. and 120° C., preferably between 75° C. and 110° C., and preferably at a pressure between 0.1 and 1.0 MPa, preferentially between 0.1 and 0.8 MPa and preferably between 0.1 and 0.5 MPa.

5. Process according to claim 1, wherein step b) is carried out at a temperature of between 70° C. and 120° C., preferentially between 75° C. and 110° C.

6. Process according to claim 1, wherein step b) is carried out at a pressure of between 0.1 and 0.8 MPa and more particularly between 0.1 and 0.5 MPa.

7. Process according to claim 1, wherein said at least one adsorbent is chosen from activated carbons, aluminas and clays.

8. Process according to claim 1, wherein the adsorption step b) uses between one and five adsorbents, preferably one or two adsorbents.

9. Process according to claim 8, wherein at least one adsorbent is an activated carbon.

10. Process according to claim 1, wherein the adsorption step b) is carried out in flow-through fixed-bed mode.

11. Process according to claim 1, comprising a separation step c) located downstream of step b), in order to obtain a separated purified diester monomer effluent and a spent aqueous solvent effluent, the separation step c) preferably using a solid-liquid separation, such as a separation by filtration, by decantation and/or centrifugation.

12. Process according to claim 1, comprising a step c*) of crystallizing the diester monomer, preferably located downstream of the adsorption step b) and in particular upstream of a separation step c), the crystallization step c*) using at least one solids production section fed by the purified monomer effluent from the adsorption step b), in order to produce an aqueous suspension of solid diester monomer.

13. Process according to claim 12, wherein the solids production section is fed by a crystallization solvent which is identical to or different from the aqueous solvent introduced into the mixing step a), the amount of crystallization solvent introduced being adjusted so that the crude diester monomer feedstock which feeds the mixing step a) represents between 1% and 75% by weight, preferentially between 5% and 45% by weight, preferably between 15% and 35% by weight, of the total weight of the mixture in said solids production section.

14. Process according to claim 12, wherein said solids production section of the crystallization step c*) is operated at a temperature of between 0° C. and 100° C., preferably between 5° C. and 80° C. and preferably between 10° C. and 70° C., and preferably at a pressure of between 0.00001 and 1.00 MPa, preferentially between 0.0001 and 0.50 MPa and preferably between 0.001 and 0.20 MPa.

15. Process according to claim 11, wherein the separation step c) is fed by the purified monomer effluent from the adsorption step b) or by the aqueous suspension of solid diester monomer obtained at the end of the crystallization step c*), in order to obtain a separated purified diester monomer effluent and a spent aqueous solvent effluent.
